(19) United States
(12) Patent Application Publication
(10) Pub. No.: US 2005/0069574 A1
(43) Pub. Date: Mar. 31, 2005

(54) Fibrous structure product comprising a discrete non-verbal cue

(75) Inventors: Scott Thomas Loughran, West Chester, OH (US); Dale James Sanko, Cincinnati, OH (US); Dea Michelle Pastwa, Cincinnati, OH (US); Luis Barnardo De la Rosa, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/672,414

(22) Filed: Sep. 26, 2003

(51) Int. Cl. A61K 9/70; A61K 35/78

(52) U.S. Cl. 424/443; 424/744; 424/764

(57) ABSTRACT

Fibrous structure products, particularly single- or multi-ply fibrous structure products, especially single- or multi-ply sanitary tissue products comprising a discrete non-verbal cue and methods for making same are provided.

### FIELD OF THE INVENTION

The present invention relates to fibrous structure products, particularly single- or multiply fibrous structure products, especially single- or multi-ply sanitary tissue products comprising a discrete non-verbal cue and methods for making same.

### BACKGROUND OF THE INVENTION

Consumers of fibrous structure products, especially sanitary tissue product, are bombarded at points of sale with numerous varieties of products that generally look and/or feel very similar. Basically, such fibrous structure products, especially sanitary tissue products are white in color and may or may not have aesthetic features within the fibrous structures themselves. Therefore, a consumer is faced with the challenge of making a purchase decision based, not on the inherent characteristics of the fibrous structure products, but rather on the packaging and/or price. For example, if two products look very similar, then the consumer, without knowing the additional features that may be present in one of the two products, may purchase the less expensive product. Only to find out later that the less expensive product is not similar in characteristics and/or performance to the other product.

Accordingly, there is a need for a fibrous structure product that comprises a discrete non-verbal cue that communicates to a consumer/purchaser of the fibrous structure product a characteristic of the fibrous structure product when the fibrous structure product is in its dry state, such as at time of purchase. Further, there is a need for a method for making such a fibrous structure product.

### SUMMARY OF THE INVENTION

The present invention fulfills the need described above by providing a fibrous structure product that comprises a non-verbal cue that communicates to a user a characteristic of the fibrous structure product.

In one aspect of the present invention, a fibrous structure product comprising a discrete non-verbal cue that communicates to a user a characteristic of the fibrous structure product when the fibrous structure product is in its dry state, is provided.

In another aspect of the present invention, a method for making a fibrous structure product comprising the steps of:
a) identifying a characteristic of a fibrous structure product to communicate to a user;
b) producing the fibrous structure product comprising a discrete non-verbal cue that communicates the identified characteristic from step a, is provided.

In even another aspect of the present invention, an article of manufacture comprising a fibrous structure product according to the present invention and an exterior package housing said fibrous structure product, wherein the exterior package is such that the discrete non-verbal cue of the fibrous structure product is capable of communicating with a user of the fibrous structure product, is provided.

In still another aspect of the present invention, a method for attracting a user to a fibrous structure product, the method comprising providing a fibrous structure product comprising a discrete non-verbal cue that communicates to the user a characteristic of the fibrous structure product when the fibrous structure product is in a dry state, is provided.

Accordingly, the present invention provides a fibrous structure product comprising a discrete non-verbal cue that communicates to a user a characteristic of the fibrous structure product; a method for making such a fibrous structure product; an article of manufacture comprising such fibrous structure product; and a method for attracting a user with such a fibrous structure product.

### BRIEF DESCRIPTION OF THE DRAWINGS

FIG. 1 is a schematic representation of one embodiment of a fibrous structure product according to the present invention, wherein the hearts comprise a green colored ply bond adhesive.

### DETAILED DESCRIPTION OF THE INVENTION

Definitions

“Cue” as used herein means a reminder and/or prompting and/or hint and/or suggestion, preferably a reminder and/or prompting, more preferably a prompting to take an action, such as to purchase a fibrous structure product.

“Non-verbal Cue” as used herein means a cue that communicates to a user through a user's senses other than hearing. For example, through non-direct senses, such as through sight (visual cues), through touch (texture cues), through smell (scent cues)

“Discrete Non-verbal Cue” as used herein means a non-verbal cue that is present on less than an entire ply of a fibrous structure product. Nonlimiting examples of discrete non-verbal cues include embossments, densified regions in a non-densified ply, non-densified regions in a densified ply, colored ply bond adhesives and perfume in the fibrous structure product.

“User” as used herein means a consumer and/or purchaser of a fibrous structure product, preferably a purchaser.

“Characteristic” as used herein means a quality or property inherent in a fibrous structure product. Aesthetics of a fibrous structure product are not a characteristic as defined herein. Nonlimiting examples of such characteristics include the presence of an ingredient, softness, absorbency, cleaning ability and/or strength, preferably the presence of an ingredient and/or softness, more preferably the presence of an ingredient.

“User recognizable characteristic” as used herein means a characteristic of a fibrous structure that a user recognizes and/or desires, preferably a characteristic of a fibrous structure that a user bases his/her purchase decision upon, such that a user would not likely purchase a fibrous structure product that did not contain such recognizable characteristic.

“Dry state” as used herein means that a fibrous structure that has not been used by a user, preferably wherein the fibrous structure is in its packaging, especially at a point of sale.

“Fiber” as used herein means an elongate particulate having an apparent length greatly exceeding its apparent width, i.e. a length to diameter ratio of at least about 10. More specifically, as used herein, “fiber” refers to papermaking fibers. The present invention contemplates the use of a variety of papermaking fibers, such as, for example, natural fibers or synthetic fibers, or any other suitable fibers, and any combination thereof. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking. In addition to the above, fibers and/or filaments made from polymers, specifically hydroxyl polymers may be used in the present invention. Nonlimiting examples of suitable hydroxyl polymers include polyvinyl alcohol, starch, starch derivatives, chitosan, chitosan derivatives, cellulose derivatives, gums, arabinans, galactans and mixtures thereof.

“Fibrous structure” as used herein means a fiber-containing structure such as a web.

“Fibrous structure product” as used herein means a fibrous structure useful as a wiping implement for post-urinary and post-bowel movement cleaning (toilet tissue), for otorhinolaryngolical discharges (facial tissue), and multi-functional absorbent and cleaning uses (absorbent towels).

“Ply” or “Plies” as used herein means an individual fibrous structure optionally to be disposed in a substantially contiguous, face-to-face relationship with other plies, forming a multi-ply fibrous structure. It is also contemplated that a single fibrous structure can effectively form two “plies” or multiple “plies”, for example, by being folded on itself.

“Basis Weight” as used herein is the weight per unit area of a sample reported in lbs/3000 ft² or g/m².

Basis weight is measured by preparing one or more samples of a certain area (m²) and weighing the sample(s) of a fibrous structure according to the present invention and/or a paper product comprising such fibrous structure on a top loading balance with a minimum resolution of 0.01 g. The balance is protected from air drafts and other disturbances using a draft shield. Weights are recorded when the readings on the balance become constant. The average weight (g) is calculated and the average area of the samples (m²). The basis weight (g/m²) is calculated by dividing the average weight (g) by the average area of the samples (m²).

Fibrous Structure Product

The fibrous structure product comprises one or more plies of fibrous structure. The fibrous structure product may be in individual sheet form or may be in roll form. If in roll form, the fibrous structure product may comprise a core upon which the one or more plies of fibrous structure are convolutely wound.

The fibrous structure product and/or plies from which the fibrous structure product is derived may be foreshortened, such as via creping, or non-forshortened, such as not creping; creped from a cylindrical dryer with a creping doctor blade, removed from a cylindrical dryer without the use of a creping doctor blade, or made without a cylindrical dryer.

The fibrous structure products of the present invention are useful in paper, especially sanitary tissue paper products including, but not limited to: conventionally felt-pressed tissue paper; through-air dried tissue paper; pattern densified tissue paper; and high-bulk, uncompacted tissue paper. The tissue paper may be of a homogenous or multilayered construction; and tissue paper products in accordance with the present invention are of a multi-ply construction. The tissue paper preferably has a basis weight of between about 10 g/m² and about 120 g/m², and density of about 0.60 g/cc or less. Preferably, the basis weight will be below about 35 g/m²; and the density will be about 0.30 g/cc or less. Most preferably, the density will be between about 0.04 g/cc and about 0.20 g/cc as measured by the Basis Weight Method described herein.

The fibrous structure product may be made with a fibrous furnish that produces a single layer embryonic fibrous web or a fibrous furnish that produces a multi-layer embryonic fibrous web.

The fibrous structure product may comprise an adhesive, such as a ply bond adhesive.

In one embodiment, the adhesive may comprise a discrete non-verbal cue.

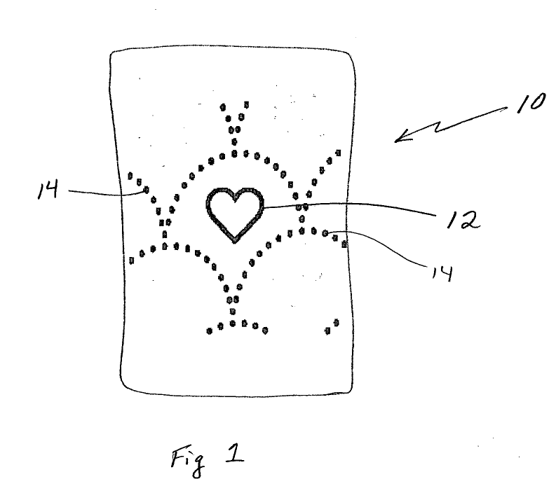

As shown in FIG. 1, an embodiment of a fibrous structure product 10 comprises a discrete non-verbal cue 12, represented by a heart, wherein the outline of the heart comprises colored ply bond adhesive. The fibrous structure product 10 further comprises additional embossments which may or may not comprise colored ply bond adhesive.

Discrete Non-Verbal Cues

The fibrous structure products of the present invention comprise a discrete non-verbal cue that communicates with a user of the fibrous structure product a characteristic of the fibrous structure product. Preferably the discrete non-verbal cue contrasts with a ply of the fibrous structure product.

The non-verbal cues may comprise visual cues, scent cues, texture cues and mixtures thereof.

The visual cues may comprise structures, color agents, text elements, pictorial elements and mixtures thereof.

Structural visual cues may comprise embossments and/or densified and non-densified regions of the fibrous structure product.

Color agents may comprise non-white color agents. Color agents may include dyes, pigments and/or other color generating agents.

Text elements may comprise letters, symbols and/or numbers.

Pictorial elements may comprise images of objects, places, people, animals and the like.

The scent cues may comprise a perfume within the fibrous structure product. The perfume may be incorporated into or applied to one or more plies of the fibrous structure product and/or may be incorporated in or applied to a core upon which the one or more plies of fibrous structure are wound. A “perfume” as used herein means a perfume that is perceivable by the sense of smell of a human.

The texture cues are cues that are perceivable by a user via the user's sense of touch. Texture cues may comprise embossments and other types of objects that interrupt the plane of a ply of fibrous structure.

Those of ordinary skill in the art know how to add discrete non-verbal cues to fibrous structure products.

Characteristics of the Fibrous Structure Product

The characteristic of the fibrous structure product may comprise an ingredient present in the fibrous structure product.

Nonlimiting examples of suitable ingredients include humectants, lotion, vitamins, perfumes, medicinal agents, therapeutic agents, virucidal agents, odor controlling agents, aroma therapy agents, softening agents and mixtures thereof.

In one embodiment, the ingredient comprises chamomile.

In another embodiment, the ingredient comprises aloe.

In yet another embodiment, the ingredient comprises vitamin E.

In even another embodiment, the ingredient comprises perfume.

The ingredient may be present in any part of the fibrous structure product. For example, in one embodiment, the ingredient may be present in the discrete non-verbal cue within the fibrous structure product, such as within a colored ply bond adhesive. Alternatively, in another embodiment, the ingredient may be present in areas of the fibrous structure product that does not include the discrete non-verbal cue.

Additional characteristics of the fibrous structure are described herein.

Communication of Characteristic

The discrete non-verbal cue may, in addition to communicating a characteristic of the fibrous structure product to a user when it is dry state, communicate a characteristic of the fibrous structure product to a user when it is in its wet state. “Wet state” as used herein means that the fibrous structure product has been subjected to a liquid, such as water.

Article of Manufacture

The present invention may comprise an article of manufacture wherein the article of manufacture comprises a fibrous structure product according to the present invention and an exterior package housing said fibrous structure product, wherein the exterior package is such that the discrete non-verbal cue of the fibrous structure product is capable of communicating with a user of the fibrous structure product.

The exterior package may itself comprise a non-verbal cue that is capable of communicating a characteristic of the fibrous structure product housed within the exterior package.

Method

The present invention further comprises a method for attracting a user to a fibrous structure product, the method comprising providing a fibrous structure product comprising a discrete non-verbal cue that communicates to the user a characteristic of the fibrous structure product when the fibrous structure product is in a dry state.

In one embodiment, the fibrous structure product is provided to the user at a point of sale, such as in a retail store.

All documents cited in the Detailed Description of the Invention are, in relevant part, incorporated herein by reference; the citation of any document is not to be considered as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A fibrous structure product comprising a discrete non-verbal cue that communicates to a user of the fibrous structure product a characteristic of the fibrous structure product when the fibrous structure product is in a dry state.

2. The fibrous structure product according to claim 1 wherein the non-verbal cue is selected from the group consisting of visual cues, scent cues, texture cues and mixtures thereof.

3. The fibrous structure product according to claim 2 wherein the visual cues are selected from the group consisting of structures, color agents, text elements, pictorial elements and mixtures thereof.

4. The fibrous structure product according to claim 3 wherein the color agents are selected from the group consisting of non-white color agents.

5. The fibrous structure product according to claim 1 wherein the characteristic of the fibrous structure product is an ingredient present in and/or on the fibrous structure product.

6. The fibrous structure product according to claim 5 wherein the ingredient is selected from the group consisting of humectants, lotion, vitamins, perfumes, medicinal agents, therapeutic agents, virucidal agents, odor controlling agents, aroma therapy agents, softening agents and mixtures thereof.

7. The fibrous structure according to claim 5 wherein the ingredient comprises chamomile.

8. The fibrous structure according to claim 5 wherein the ingredient comprises aloe.

9. The fibrous structure according to claim 5 wherein the ingredient comprises vitamin E.

10. The fibrous structure according to claim 5 wherein the ingredient comprises perfume.

11. The fibrous structure according to claim 5 wherein the non-verbal cue comprises the ingredient.

12. The fibrous structure product according to claim 1 wherein the characteristic of the fibrous structure product comprises a user recognizable property of the fibrous structure product.

13. The fibrous structure product according to claim 12 wherein the user recognizable property comprises softness.

14. The fibrous structure product according to claim 12 wherein the user recognizable property comprises cleaning ability.

15. The fibrous structure product according to claim 12 wherein the user recognizable property comprises absorbency.

16. The fibrous structure product according to claim 12 wherein the user recognizable property comprises strength.

17. The fibrous structure product according to claim 1 wherein the fibrous structure product comprises a fibrous structure comprising an adhesive.

18. The fibrous structure product according to claim 17 wherein the adhesive comprises the non-verbal cue.

19. The fibrous structure product according to claim 1 wherein the fibrous structure product is a single- or multi-ply sanitary tissue product.

20. The fibrous structure product according to claim 1 wherein the non-verbal cue communicates with the user of the fibrous structure product a characteristic of the fibrous structure product when the fibrous structure is in a wet state.

21. An article of manufacture comprising a fibrous structure product according to claim 1 and an exterior package housing said fibrous structure product, wherein the exterior package is such that the discrete non-verbal cue of the fibrous structure product is capable of communicating with a user of the fibrous structure product.

22. A method for attracting a user to a fibrous structure product, the method comprising providing a fibrous structure product comprising a discrete non-verbal cue that communicates to the user a characteristic of the fibrous structure product when the fibrous structure product is in a dry state.
